(19) United States
(12) Patent Application Publication
(10) Pub. No.: US 2024/0390686 A1
(43) Pub. Date: Nov. 28, 2024

(54) MEDICAL DEVICE SYSTEM INCLUDING A PROGRAMMABLE IMPLANTABLE MEDICAL DEVICE SUCH AS A NEUROSTIMULATOR AND METHOD FOR OPERATING SAME

(71) Applicant: BIOTRONIK SE & Co. KG, Berlin (DE)

(72) Inventors: Pamela Shamsie Victoria Riahi, Portland, OR (US); Sean Slee, Tigard, OR (US)

(21) Appl. No.: 18/694,140

(22) PCT Filed: Oct. 6, 2022
(86) PCT No.: PCT/EP2022/077797

§ 371 Date: Mar. 21, 2024

Related U.S. Application Data
(60) Provisional application No. 63/253,636, filed on Oct. 8, 2021.

(30) Foreign Application Priority Data
Oct. 25, 2021 (EP) 21204481.2

(51) Int. Cl. A61N 1/372; A61N 1/05; A61N 1/36; G16H 10/20; G16H 80/00

(52) U.S. Cl. CPC A61N 1/37282; A61N 1/0551; A61N 1/36062; A61N 1/36132; A61N 1/36139; A61N 1/37247; A61N 1/37264; G16H 10/20; G16H 80/00

(57) ABSTRACT

A medical device system (1) and a method for operating a medical device system are de-scribed. The medical device system, comprises: a programmable implantable medical device (3), configured for providing stimulations to a patient (11) in accordance with programmed stimulation parameters; a patient device (5), comprising a first interface (19) configured for communication and interaction with the patient and a second interface (23) configured for submitting data for programming the implantable medical device, and a clinician device (7), comprising a third interface (27) configured for communication and interaction with a clinician (29) and a fourth interface (33) configured for data communication with the patient device, wherein, for programming the implantable medical device, the patient device is configured to automatically pose at least one question to the patient via the first interface, and to automatically decide upon an action according to at least one feedback from the patient entered via the first interface and to submit the adjusted stimulation parameter to the implantable medical device, and wherein the patient device and the clinician device are further configured to provide an option to the patient to selectively establish a communication between the patient and the clinician.

The present invention relates to a medical device system including, inter-alia, a programmable implantable medical device. Furthermore, the present invention relates to a method for operating a medical device system.

There are various medical devices which may be implanted into a body of a patient. Such medical devices may for example provide stimulations to a body part of the patient. For example, the medical device may apply physical stimulations which induce physiological reactions within the body. As an example, such stimulation may be an electric current pulse applied to tissue or cells of the body. Such voltage pulse may induce reactions due to influencing for example electric potentials at the tissue or cells. As an alternative, an implanted medical device may provide chemical stimulations inducing physiological reactions. As an example, chemical compounds may be injected into the patient's body in a controlled manner.

A specific type of implantable medical device is a neurostimulator. Such neurostimulator may be used in pain therapy or in treating neurological dysfunctions. For such purpose, the neurostimulator may apply electric pulses to nerves, for example nerves in the spinal cord.

Implantable medical devices may be programmable. Accordingly, functionalities of such medical devices may be varied and/or adapted in reaction to for example changing environmental and/or physiological conditions by specifically programming parameters within the medical device. In reaction to the programming, the medical device may then for example provide stimulations in a varied manner, i.e. for example with a varied amplitude, intensity, time pattern, etc. Typically, the programming may be established using an external device which may communicate with the implanted medical device, preferably via wireless data communication.

For example, for a programmable neurostimulator, parameters such as an electric voltage, an electric current, a time profile of a single electric pulse and/or a time profile of several pulses comprised in a pulse sequence may be varied in order to adapt applied stimulations to for example actual pain sensations of the patient.

Conventionally, programming of a programmable implantable medical device is executed by a clinician, i.e. a medical expert, upon setting up the medical device. For such purpose, the clinician may, in a personal session, communicate with the patient in order to get the patient's feedback about sensations resulting upon varying stimulation characteristics applied by the medical device. In such personal session, the clinician typically poses a set of questions to the patient in order to acquire a detailed feedback from the patient, optionally also taking into account additional data such as the patient's current physiological condition and/or health status as well as possibly other environmental conditions. Taking into account such feedback and optional additional data, the clinician may then specifically select parameters to be programmed to the implantable medical device and transmit such parameters to the medical device using for example a wirelessly coupled programming device.

However, such programming procedure may be time-consuming, subject to human error and/or method inconsistency and/or stressful for both, the clinician as well as the patient.

Furthermore, an effectiveness of stimulations provided by the medical device may change over time. For example, a location of implanted electrodes of the medical device may change over time resulting in stimulations applied by such electrodes effecting the patient's tissue or cells in a different way and being therefore sensed by the patient differently. Furthermore, the patient's physiological sensitivity to stimulations may change over time. Accordingly, the programming procedure may have to be repeated from time to time, thereby again requiring time and efforts by both, the clinician and the patient.

Approaches have been developed in which the patient may effect programming of the medical device without requiring support by the clinician.

For example, U.S. Pat. No. 7,386,348 B2 discloses a fully automated computer-controlled system provided for adjustment of neurostimulation implants used in pain therapy and in treating neurological dysfunctions. The system includes a patient interactive computer and a universal transmitter interface being capable of stimulating any type of implanted neurostimulator and devices by imitating programming codes. The patient interacts with the system through the patient interactive computer. Accordingly, there is an option for the patient to directly reprogram the neurostimulation implant without requiring support of the clinician.

However, it has been observed that patients may be hesitant in programming their implanted medical device. Particularly, patients of advanced age or of reduced mental fitness may have reservations in using a computer-controlled system which, on the one hand, for them, may appear to be complex and which, on the other hand, may directly negatively influence their physiological status upon programming the implanted device in an undesired manner.

Accordingly, it may be an object of the present invention to provide an approach for medical device systems including programmable implanted devices and for using and operating such medical devices systems, the approach enabling better acceptance by patients. Furthermore, it may be an object to provide a computer program product implementing such approach upon being executed on a processor of a medical device system and a computer readable medium comprising such computer program product stored thereon.

Such objects may be met with the subject-matter of the independent claims. Advantageous embodiments are defined in the dependent claims as well as the corresponding specification and FIGURES.

According to a first aspect of the present invention, a medical device system is proposed, the medical device system comprising a programmable implantable medical device, a patient device and a clinician device. The programmable implantable medical device is configured for providing stimulations to a patient in accordance with programmed stimulation parameters. The patient device comprises a first interface configured for communication and interaction with the patient and a second interface configured for submitting data for programming the implantable medical device. The clinician device comprises a third interface configured for communication and interaction with a clinician. For programming the implantable medical device, the patient device is configured to automatically pose at least one question to the patient via the first interface, and to automatically decide upon an action according to at least one feedback from the patient entered via the first interface and to submit the adjusted stimulation parameter to the implantable medical device. Furthermore, the patient device and the clinician device are configured to provide an option to the patient to selectively establish a communication between the patient and the clinician.

According to a second aspect of the invention, a method for operating a medical device system is proposed. Therein, the medical device system comprises a programmable implantable medical device, a patient device and a clinician device, each having the characteristics as defined above with respect to the first aspect of the invention. The method comprises at least the following steps, preferably in the indicated order:
automatically posing at least one question to the patient via the first interface of the patient device,
automatically decide upon an action according to at least one feedback from the patient entered via the first interface and
submitting the adjusted stimulation parameter to the implantable medical device for programming the implantable medical device,
   wherein, during operation of the patient device, an option is provided to the patient to selectively establish a communication between the patient and the clinician.

According to a third aspect of the invention, a computer program product is proposed, the computer program product comprising computer readable instructions which, upon being executed by a processor in a medical device system, instruct the processor to either controlling the medical device system to implement the method according to an embodiment of the second aspect of the invention, or controlling the medical device system to adopt its configurations as defined herein with regard to an embodiment of the first aspect of the invention.

According to a fourth aspect of the invention, a computer readable medium is proposed, the computer readable medium comprising the computer program product according to an embodiment of the third aspect of the invention stored thereon.

Ideas underlying embodiments of the present invention may be interpreted as being based, inter alia, on the following observations and recognitions.

Briefly summarised in a non-limiting manner, embodiments of the present invention relate to a medical device system and a method for operating such system, in which a patient, additionally to having an option to directly program stimulation parameters of an implantable medical device using a specific patient device, has the option to selectively establish a data communication via which data may be communicated between the patient device and a clinician device. Due to such additional option, on the one hand, the patient may for example directly communicate with a clinician, i.e. the patient may use the patient device for communicating with the clinician device handled by the clinician, or the patient may trigger a request to the clinician to be called back (audio or video call) by the clinician or medical representative on another device (e.g. personal phone, computer, etc.). Accordingly, in case of any doubts or uncertainties, the patient may acquire help directly by the clinician. On the other hand, as there is a direct data communication between the clinician device and the patient device, the clinician may directly access data available within the patient device in order to support the patient in correctly using the patient device or to even submit data for programming the implantable medical device directly to the patient device. As a result of such additional option, hesitant patients may be supported in using the medical device system. Thereby, acceptance of such system may be improved as for example the patients know that, in case of any problems or doubts, they can directly inquire support by a clinician via the optional data communication between the patient device and the clinician device.

According to an embodiment of the invention, the clinician device comprises a fourth interface. For establishing the communication between the patient and the clinician, the patient device is configured to setup a data communication to an external server, whereupon the external server is configured to setup a data communication to the clinician device. Alternatively, the data communication may be established from the opposite direction, i.e. first the clinician device is configured to setup a data communication to an external server, whereupon the external server is configured to setup a data communication to the patient device. The data communication between the patient device and the clinician device is carried out via the external server.

According to an embodiment, the external server may have an integrated support center for clinicians and patients of the implantable medical device.

According to an embodiment of the present invention, the feedback from the patient may include information provided by the patient in response to the question posed (e.g. related to the physical and/or mental status of the patient, as for instance pain level, location of pain, the patient's subjective level of well-being). Alternatively or in addition, the information is provided by the programmable implantable device and transmitted to the patient device (e.g. evoked compound action potentials (eCAP) measurements as a one-time test per session, or automatically recorded by the device on a regular basis; lead impedance measurements, charging state of the implantable device, etc.), or the information is provided by another implantable or non-implantable device of the patient (e.g. cardiac pacemaker which measures and provides information on the heart rate or Heart Rate Variability (HRV), a glucose sensor which provides the blood sugar level, etc.).

In the following, characteristics of embodiments of the present invention will be described in more detail. Therein, a focus will be set on embodiments in which the implantable medical device is a neurostimulator. However, it is to be noted that alternatively or additionally, other implantable medical devices may be included in the proposed medical device system and its operating method as described herein.

The implantable medical device may be configured for being implanted into a body of a patient. For example, the medical device may be implanted subcutaneously. Accordingly, a size, geometry, materials and/or functionalities of the medical device may be specifically configured for such implantation purposes.

The medical device, particularly when implemented as a neurostimulator, may comprise one or more electrodes for transmitting and/or receiving electrical signals to or from body parts, such as tissue, nerves or cells, of the patient. Furthermore, the medical device may comprise a circuitry for generating and outputting electrical signals and, optionally, for modifying characteristics of such electrical signals such as their amplitude, intensity, duration, time intervals between successive signals, etc., in order to thereby modify characteristics of stimulations provided by the electrical signals to the patient's body. Alternatively or additionally, the medical device may comprise circuitry for receiving and/or evaluating electrical signals such that electrical signals received by one or more of the electrodes may be analysed in order to determine physiological conditions within the patient's body.

The implantable medical device is programmable. Accordingly, characteristics of stimulations provided by such medical device may be modified in accordance with stimulation parameters programmed into such device. For such purpose, the medical device typically comprises a circuitry and/or a processor which may modify output stimulation signals in accordance with the programmed stimulation parameters. Furthermore, the medical device typically comprises data memory for storing the stimulation parameters. Furthermore, one or more data interfaces are typically comprised in the implantable medical device for receiving and/or transmitting data between this device and one or more external devices. The data interfaces may be configured for wireless data transmission.

The patient device is generally separate to the implantable medical device and is generally non-implantable. The patient device may be configured for being operated by a patient, i.e. by a person which is not specifically trained in medical and/or technical affairs. The patient device may be a portable device or handheld device such as a smartphone, a tablet, a laptop, etc. The patient device may be a multi-purpose device on which a specific software or app is installed for implementing the functionalities defined for the patient device of the medical device system described herein. Alternatively, the patient device may comprise specific hardware and/or software developed for its required functionalities. The patient device may be programmable. Accordingly, the patient device may have a processor for processing data and a memory for storing data. The patient device is configured to, on the one hand, communicate and interact with the patient and, on the other hand, communicate and interact with the implantable medical device.

For such purpose, the patient device comprises a first interface via which data may be input and/or output from and to the patient. Such first interface may be a human machine interface (HMI). Particularly, the first interface may have input functionalities such that the patient may input data. For example, the first interface may comprise a keyboard for inputting instruction data via mechanical actuation, a microphone for inputting data via acoustic instructions, a camera for inputting data using visual instructions or any other instruction receiving device. Furthermore, the first interface may have output functionalities such that data may be output to the patient. For example, the first interface may be a screen for outputting data visually or a loudspeaker for outputting data acoustically. In a preferred implementation, the first interface may be a touch screen via which data may be both input and output.

Furthermore, the patient device comprises a second interface via which data may be input and/or output from and to the implantable medical device. Such second interface may be a machine-machine interface (MMI). Particularly, the second interface may have output functionalities such that for example stimulation parameters entered by the patient into the patient device may be forwarded to the implantable medical device. Furthermore, the second interface may have input functionalities such that for example data indicating a current condition of the implantable medical device and/or data sensed by such medical device may be transmitted to the patient device. The second interface may preferably be configured for wireless data communication. Furthermore, as the patient device is generally handled by the patient and is therefore in close proximity to the implanted medical device, the second interface may be configured for short range data communication such as for example near field communication (NFC).

The clinician device is generally separate and remote to both, the implanted medical device as well as the patient device. The clinician device may be configured for being operated by a clinician, i.e. by a person which is specifically trained in medical affairs and which has generally received a specific training in handling technical devices of medical device systems. Similarly to the patient device, the clinician device may be a portable or handheld device such as a smartphone, a tablet, a laptop, etc. Furthermore, similarly to the patient device, the clinician device may be of a multi-purpose nature or may be specifically developed for the purposes described herein. The clinician device may be programmable. Accordingly, the clinician device may have a processor for processing data and a memory for storing data. The clinician device is configured to, on the one hand, communicate and interact with the clinician and, on the other hand, communicate data with the patient device.

For such purpose, the clinician device comprises a third interface via which data may be input and/or output from and to the clinician. Such third interface may be a human machine interface (HMI). Particularly, the third interface may have input functionalities such that the clinician may input data. The third interface may be implemented in a same or similar manner and/or with same or similar functionalities as the first interface of the patient device.

Furthermore, the clinician device comprises a fourth interface via which data may be input and/or output from and to the patient device. The fourth interface can be configured to establish a data communication to an external server, wherein the external server establishes a data communication to the patient device. In such embodiment, communication between patient device and clinician device is carried out via the external server. Such fourth interface may be a machine-machine interface (MMI). Particularly, the fourth interface may have input functionalities such that for example data available in the patient device may be forwarded to and received by the clinician device. For example, input stimulation parameters received by the patient and stored in the patient device may be transmitted via the fourth interface to the clinician device. Additionally, other data received by the patient device for example via its first interface may be communicated to the clinician device. Furthermore, the fourth interface may have output functionalities such that for example data available in the clinician device may be forwarded to the patient device. For example, the clinician may input data or parameters into the clinician device which may then be forwarded to the patient device. The second interface may be configured for wired data communication, wireless data communication or a mixture of both. For example, as the clinician device is typically arranged remotely from the patient device, both devices may be configured for communicating via a network such as the Internet. For the data communication with the clinician device, the patient device may have an additional interface referred to herein as fifth interface.

The patient device is configured for enabling the patient to input data which may then optionally be pre-processed and based on which stimulation data may then be generated and submitted to the implanted medical device for programming this device. For such purpose, the patient device is configured to pose one or more questions to the patient via the first interface. As indicated in further detail below, the questions may be posed in a way such as to be understood and responded by the patient easily. The patient may respond to the questions by entering data as a feedback. Taking into account such feedback, the patient device may then adjust at least one of the stimulation parameters to be programmed in the medical device.

Additionally, to such functionality in which the patient may use the patient device to autonomously program the implanted medical device, the patient device is further configured for another functionality in which it may establish a data communication with the clinician device. Such option of establishing the data communication with the clinician device may be implemented in a simple and intuitive way such that the patient, in case of any doubts or uncertainties while programming the medical device, may easily and quickly contact a clinician via the clinician device in order to inquire support by the clinician. Accordingly, having such option of contacting the clinician, an acceptance of the patient upon using the entire medical device system may be enhanced.

According to an embodiment of the medical device system, the patient device and the clinician device are further configured to provide an option to the patient to communicate with a clinician. In an embodiment of the invention, the patient device and the clinician device are configured to provide an option to the patient to communicate with a clinician by at least one of
establishing a data communication between the patient device and the clinician device,
providing a notification on the third interface to the clinician to call back the patient.

Analogously, with regard to an embodiment of the method for operating the medical device system, upon specifically actuating the patient device by the patient, an option is provided to the patient to communicate with a clinician. In an embodiment, upon specifically actuating the patient device by the patient, an option is provided to the patient to communicate with a clinician by at least one of
establishing a data communication between the patient device and the clinician device,
providing a notification on the third interface to the clinician to call back the patient.

According to embodiments of the present inventive method, the clinician device comprises a fourth interface,
and wherein for establishing the communication between the patient and the clinician,
   a data communication is set up between the patient device and an external server, whereupon a data communication is set up between the external server to the clinician device via the fourth interface, or
   a data communication is set up between the clinician device and an external server via the fourth interface, whereupon a data communication is set up between the external server to the patient device,
      wherein the data communication between the patient device and the clinician device is carried out via the external server.

In other words, the patient device and the clinician device may be specifically adapted and used to establish a direct communication between the patient and the clinician. Accordingly, in cases where the patient doubts for example how to correctly use the patient device or how to correctly answer questions posed by the patient device, the patient may inquire support by directly contacting the clinician via the patient device. The communication between the patient and the clinician may be established as voice communication using the data communication implemented between the patient device and the clinician device. In such cases, the clinician may communicate with, i.e. for example talk to, the patient based on the clinician's medical expertise and provide some medical advice.

The communication between the patient device and clinician device may be carried out via an external server. According to an embodiment, the option provided to the patient to communicate with a clinician comprises triggering a notification on the interface of the clinician device, informing the clinician to call back the patient. According to an embodiment, upon the patient uses the option to communicate with the clinician, an alert in the remote server is triggered regarding the clinician to call back the patient. The remote server initiates a notification on the clinician device. According to an embodiment, the clinician may call back the patient on the patient device or any other personal device of the patient having communication capabilities (e.g. personal tablet, phone/computer).

According to embodiments of the invention, the clinician device may be a specific programmer device, or any device of the clinician (e.g. tablet or personal phone/computer) having the capability to communicate with the patient device and/or the external server.

Additionally or alternatively, according to an embodiment of the medical device, the patient device and the clinician device are further configured to provide an option to the clinician device to establish access to data stored in the patient device using the data communication between the patient device and the clinician device. Analogously, with regards to an embodiment of the method for operating the medical device system, during operation of the patient device, an option is provided to the clinician to access to data stored in the patient device using the data communication between the patient device and the clinician device.

Accordingly, additionally or instead of simply talking to the patient, the clinician may use the data communication between the patient device and the clinician device to have direct access to data in the patient device. Therefore, the clinician may for example verify and/or correct stimulation data previously input by the patient. Alternatively, the clinician may analyse data acquired by the implanted medical device and transmitted to the patient device with regard to characteristics of the stimulations provided by the medical device and/or characteristics of physiological conditions as sensed for example by the medical device. Accordingly, using such data access, the clinician may use the clinician device and support the patient in correctly programming the implantable medical device using the patient device.

According to an embodiment, the patient device comprises a clinician contact button configured for
establishing the data communication between the patient device and the clinician device,
or prompting a message to the clinician device for calling back the patient,
   upon the clinician contact button being actuated by the patient.

Expressed differently, the patient device is specifically adapted for providing an option to the patient to very simply establish the data communication to the clinician device by simply actuating a specific button referred to herein as clinician contact button. Such button may be a physical button or switch which is provided at the patient device and which may be actuated for example by a mechanical pushing action. Alternatively, the button may be a virtual button which is for example displayed on a touchscreen of the patient device and which may be actuated by touching this button. The clinician contact button may be accessible during the entire operation of the patient device, i.e., the button may be visible to the patient and may be actuated by the patient whenever the patient uses the patient device. Accordingly, being aware of the presence of such clinician contact button, the patient may be less hesitant in using the patient device for programming the implanted medical device.

Generally, the patient device may be configured such that the data communication between the patient device and the clinician device may exclusively be established upon the patient's initiative. In other words, as long as the patient does not explicitly authorise establishing such data communication, there is no link between the patient device and the clinician device and therefore no data communication between both devices. Accordingly, the patient device may be prevented from any unintended manipulations from external devices, independent of whether such external device is the clinician device or any other device. Thereby, operation safety of the entire medical device system may be enhanced as unauthorized manipulations by external devices may be prevented.

According to an embodiment, the patient device is configured to automatically pose the at least one question in a manner mimicking an inter-human communication.

In other words, the patient device may ask questions to the patient in a way which is similar to a communication between humans. Therein, the questions may be posed e.g. in full phrases, preferably in a language which may be easily understood by the patient. For example, such questions may not include specific medical terms with regard to the patient's current physical condition but, instead, preferably includes asking the patient about his current body sensations. Preferably, the questions are posed such that the patient may simply answer with “yes” or “no”. Alternatively or additionally, at least some of the questions may be posed such that the patient may simply answer by selecting a specific one of proposed answers and/or by indicating a number within a range ranging for example from “1” (representing “completely true”) to “10” (representing “completely wrong”). For example, the questions may be posed by a computer program, which is sometimes referred to as a bot, imitating questions as they are usually posed by a clinician in an interview with the patient. Such computer program may be based on artificial intelligence and may be trained based on real inter-human communications between clinicians and patients.

According to an embodiment, the patient device is configured to automatically pose several questions to the patient following a predefined protocol.

Accordingly, there may be a protocol including a plurality of questions. The questions are preset and may correspond to the questions which are typically posed by a clinician upon interviewing the patient for initially programming the medical device system or re-programming such system. Thus, the questions are posed independent on the actual feedback of the patient. Accordingly, the questions may be posed in a standardised manner and the feedback of the patient may be analysed and evaluated in a standardised manner as well.

As an alternative, the patient device may be configured to automatically pose several questions to the patient in manner in which the questions themselves depend on the patient's feedback to preceding questions. Such posing of questions may be referred to as “interactive”. While such approach of posing questions may require a more sophisticated implementation in the patient device, it may help the patient in providing the feedback and simplifying the use of the patient device.

According to an embodiment, the implantable medical device comprises at least one implantable electrode. Therein, the medical device system is configured for automatically determining a position of the implantable electrode relative to the patient's body or relative to a position of a neighboring electrode. The patient device is configured to automatically posing the at least one question to the patient in dependence of the determined position of the implantable electrode. Additionally or alternatively, the patient device is configured to automatically decide upon an action according to at least one feedback from the patient in dependence of the determined position of the implantable electrode.

Furthermore, according to embodiments of the invention, the action decided upon by the patient device is at least one of:
an adjustment of at least one stimulation parameter,
decision to not adjust any stimulation parameter,
initiating a call with the clinician device,
providing a notification to the clinician to call back the patient on the third interface,
pose another question to the patient via the first interface.

The implantable electrode may be arranged e.g. on an implantable lead. Preferably, several electrodes are arranged on a same lead and/or several electrodes may be arranged on a multiplicity of leads. The electrodes may be configured for being arranged in direct contact with tissue, nerves or cells in a body part such that electric pulses may be transmitted to the tissue, nerves or cells.

The medical device system may then automatically determine an actual position of the one or more electrodes relative to the patient's body. For such purpose, for example stimulations may be emitted via an electrode and an information about the position of the electrode may be determined taking into account detected physiological reactions resulting from such stimulations. In case of several electrodes being present, the medical device may automatically determine the position of the electrodes relative to each other. For such purpose, for example electric signals may be emitted by an electrode and an information about the position of a neighbouring electrode may be determined taking into account signals measured at the neighbouring electrode resulting from such emitted signals.

The acquired information about the relative position of one or more electrodes is subsequently be used by the patient device (and/or transmitted by the patient device to an external server for data processing, then communicated back to the patient device), upon automatically posing the at least one question to the patient. For example, the questions may be varied taking into account the position of the electrode(s) relative to specific body parts or relative to each other. Therein, for example some questions may or may not be posed when the electrodes are arranged in a predefined configuration. As an example, specific questions about the patient's sensations may only be posed when it is determined that an electrode has meanwhile been displaced from its original position within the patient's body, in order to determine whether the patient senses any differences resulting from such electrode displacement.

Additionally or alternatively, the acquired information about the relative position of one or more electrodes is subsequently be used by the patient device (and/or transmitted by the patient device to an external server for analysis), upon automatically adjusting the at least one stimulation parameter according to at least one feedback from the patient. In other words, upon adjusting the stimulation parameters, it may for example be taken into account that the relative position of one or more electrodes has meanwhile been changed since for example a preceding setup of the medical device system.

According to an embodiment, the medical device system comprises at least one body position sensor. The medical device system is configured for automatically determining a position of the patient's body based on signals provided by the body position sensor. The patient device is configured to automatically posing the at least one question to the patient in dependence of the determined position of the patient's body. Additionally or alternatively, the patient device is configured to automatically adjusting the at least one stimulation parameter according to at least one feedback from the patient in dependence of the determined position of the patient's body.

The body position sensor may include one or more sensing units which may provide signals based on which information about a current position of the patient's body may be determined. For example, such body position sensor may include one or more acceleration sensing units arranged at specific locations at the patient's body such that, based on signals provided by such sensing units, an information about a relative location of respective body parts may be determined. Accordingly, using the body position sensor, the current position of the patient's body may be analyzed, i.e. it may for example be determined whether the patient is currently standing, sitting upright on a stool, sitting relaxed in an armchair or is lying.

Similarly as indicated with regards to the preceding embodiment, such information about the current position of the patient's body may subsequently be used upon automatically posing the at least one question to the patient. Therein, the questions may be varied taking into account for example whether the patient is currently standing, sitting or lying. Additionally or alternatively, the acquired information about the current position of the patient's body may subsequently be used upon automatically adjusting stimulation parameters. Therein, stimulation parameters may be set taking into account whether the patient is currently standing, sitting or lying.

According to an embodiment, the patient device is configured to submit the adjusted stimulation parameter to the implantable medical device exclusively upon the patient having authorized such submitting.

In other words, in a session of programming or re-programming the implantable medical device, the patient device may pose the questions to the patient and may adapt the stimulation parameters in accordance with the feedback of the patient. Additionally or alternatively, the stimulation parameters may be changed by the clinician upon a data communication having been established between the patient device and the clinician device. However, the adjusted stimulation parameters may not be directly transmitted to the implanted medical device. Instead, the submission of such adjusted stimulation parameters is specifically be prevented until being explicitly authorized by the patient.

Accordingly, the patient can always decide independently whether or not or when functionalities of the medical device system shall be modified. Particularly, it may be prevented that modifications are applied in situations which are undesired by the patient. Furthermore, any unintended, improper or even abusive manipulations of the implanted medical device may be prevented, as no external device or party may modify the functions of the medical device without agreement and authorization by the patient.

According to an embodiment, the patient device comprises a stop stimulation button configured for immediately instructing the implantable medical device to stop providing stimulations to the patient upon the stop stimulation button being actuated by the patient.

Similarly to the clinician contact button, the stop stimulation button may be a physical or virtual button or switch. This stop stimulation button may be actuated by the patient in order to immediately interrupt the provision of stimulations by the implanted medical device. Accordingly, actuating such stop stimulation button may be a kind of emergency reaction for example in cases where stimulations provided by the implanted medical device result in undesired sensations by the patient.

Having access to such stop stimulation button may further enhance the patient's acceptance of the entire medical device system. Furthermore, an actuation of the stop stimulation button may be reported to the clinician, thereby possibly indicating that the patient requires support in operating the medical device system.

The functionalities described herein for embodiments of the medical device system or for embodiments of the methods for operating such medical device system may be implemented using software and/or hardware. The software may be provided as a computer program product in accordance to embodiments of the third aspect of the invention. Computer readable instructions of such computer program product may be executed by one or more processors in the medical device system. For example, part of these instructions may be executed by a processor included in the patient device, part of the instructions may be executed by a processor included in the clinician device and/or part of the instructions may be executed by a processor included in the implanted medical device.

The computer program product may be stored on any computer readable medium in accordance with the fourth aspect of the invention. The computer readable medium may be portable. For example, the computer readable medium may be a CD, a DVD, a flash memory or similar memory devices. Alternatively, the computer readable medium may be part of a computer or server. Such computer or server may be located external to the medical device system. The computer or server may be part of a data cloud. The computer readable medium may be downloaded from such computer or server for example via a network. The network may be the Internet.

It shall be noted that possible features and advantages of embodiments of the invention are described herein with respect to various embodiments of medical device system, on the one hand, and with respect to various embodiments of a method for operating such medical device system, on the other hand. One skilled in the art will recognize that the features may be suitably transferred from one embodiment to another and features may be modified, adapted, combined and/or replaced, etc. in order to come to further embodiments of the invention.

In the following, advantageous embodiments of the invention will be described with reference to the enclosed drawing. However, neither the drawing nor the description shall be interpreted as limiting the invention.

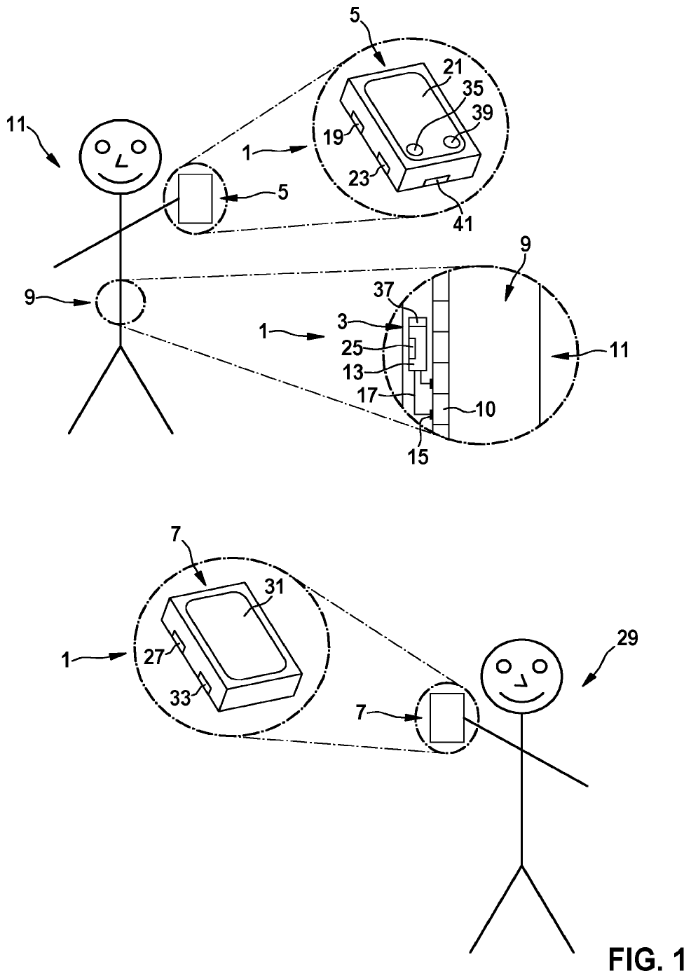

FIG. 1 shows a medical device system according to an embodiment of the present invention.

The FIGURE is only schematic and not to scale. Same reference signs refer to same or similar features.

FIG. 1 shows an exemplary medical device system 1 including a programmable implantable medical device 3, a patient device 5 and a clinician device 7.

The medical device 3 may be implanted in a body 9 of a patient 11. In the present example, the medical device 3 is a neurostimulator 13. Accordingly, the entire medical device system 1 may be interpreted as neurostimulation system. The neurostimulator 13 comprises several electrodes 15 arranged at implantable leads 17. The electrodes 15 may be implanted in contact with nerves of the body 9 for example at a spine 10 of the patient 11.

The patient device 5 includes a first interface 19 forming an HMI for communication and interaction with the patient 11. In the present example, the patient device 5 is implemented by executing an app on a personal mobile device such as a smart phone of the patient 11. In this example, the first interface 19 comprises a touchscreen 21 via which information may be output to the patient 11 as well as via which data may be input by the patient 11.

Furthermore, the patient device 5 includes a second interface 23 for submitting data to the implantable medical device 3 for programming the latter. Such second interface 23 may establish a wireless data communication between the patient device 5 and the implanted medical device 3. Accordingly, a corresponding interface 25 may be provided in the implanted medical device 3. Both, the second interface 23 in the patient device 5 and the corresponding interface 25 in the medical device 3 may be configured for short range data communication.

The clinician device 7 includes a third interface 27 for communication and interaction with a clinician 29. Similarly to the patient device 5, such clinician device 7 may be implemented for example by executing an app on a personal mobile device of the clinician 29. The third interface 27 may again comprise for example a touchscreen 31.

The clinician device 7 further includes a fourth interface 33 for data communication with the patient device 5. The patient device 5 may have an additional fifth interface 41 for establishing the data communication. Such data communication may be established for example via a network. The network may be wired or wireless. Particularly, the data communication between the clinician device 7 and the patient device 5 may be web-based.

In order to program the implantable medical device 3, the patient device 5 may automatically pose one or more questions to the patient 11 via the first interface 19. As a feedback, the patient 11 may answer to these questions. Based on such feedback, the patient device 5 may then automatically adjust at least one stimulation parameter and submit the adjusted stimulation parameter to the implanted medical device 3 via the second interface 23.

The patient device 5 further includes an option for selectively establishing a data communication with the clinician device 7. For this purpose, the patient device 5 comprises a clinician contact button 35. Upon actuating this clinician contact button 35, the data communication with the clinician device 7 is established and the patient 11 may for example use the established data communication with the clinician device 7 in order to directly communicate with the clinician 29. Furthermore, the clinician 29 may use the established data communication in order to have access to data in the patient device 5 and, optionally, may even modify such data.

Furthermore, the medical device system 1 includes at least one body position sensor 37 included for example in the implanted medical device 3. Using this body position sensor 37, it may for example be determined whether the patient 11 is currently standing, sitting or lying. This may be taken into account upon adjusting stimulation parameters.

As another feature, the patient device 5 comprises a stop stimulation button 39 for immediately stopping any provision of stimulations to the patient 11.

In the following paragraphs, some possible characteristics or features of embodiments of the medical device system 1 and of a corresponding operation method will be described partly with a wording which may slightly deviate from the wording used in the above introductory section and in the claims, in order to further enhance an understanding of possible implementations.

In an embodiment, a method for automatic management of implanted neurostimulation systems is described.

Initial programming or follow-up reprogramming of neurostimulation systems, such as an implanted SCS system, is typically a time consuming, yet crucial task that involves repetitive interactions between a clinical field specialist and the implanted patient. Usually, a standard workflow is established for the clinical field specialist to navigate system programming using the patient's feedback.

It is proposed herein to automatize this process using a digital system that takes the patient's feedback as input and automatically programs or adjusts the therapy according to a pre-defined workflow. This system can take the form of an additional module in a patient's personal remote software, or a separate app with secure online access (e.g. a web app). The system has secure access to the neurostimulator parameters and has rights to modify them within a safe window.

The patient triggers a programming session by starting the app and entering the type of session (e.g. initial programming, follow up due to unsatisfactory pain relief). The patient digitally enters their area and intensity of current pain through typical body map and NRS scale used in clinical settings. The system then starts stimulation according to the pre-defined workflow, taking into account previous parameters if it is a follow-up, with patient-authorized stimulation onset and increases. It records paresthesia threshold when necessary by asking to increase stimulation and asking if the patient felt something. At the end of the programming session, the system programs the new parameters as a result of the followed workflow and requires the patient to wait at least a pre-defined period of time (e.g. 1 day) before evaluating the efficacy of the new programs and triggering a new programming session. At any time, the patient can decide to call a clinical field specialist who can remotely take over the programming session just as it would be typically done in-office.

Initial programming or follow-up reprogramming of neurostimulation systems, such as an implanted SCS system, is always a time consuming, yet crucial task that involves repetitive interactions between a clinical field specialist and the implanted patient. Typically, a workflow is established for the clinical field specialist to navigate system programming using the patient's feedback. The workflow typically consists of starting stimulation with a standard electrode configuration, measuring perception threshold, and adjusting electrode configuration according to patient's paresthesia mapping with the areas of pain. Then amplitude is adjusted to a fraction of the perception threshold.

At therapy initiation after implant, multiple programs with different electrode configurations and/or different therapy types (e.g. traditional supra-threshold and low frequency, higher frequency sub-perception, multi-phase sub-perception) are typically programmed for the patient, so that they can switch between them once out of the clinic, using their personal system remote. They can also adjust amplitude for each program.

However, a significant proportion of patients undergo habituation after a certain period of time using the neurostimulator, or implanted electrodes migrate within the body, which results in unsatisfactory pain relief with all available programs. In those cases, follow-up programming sessions are scheduled to re-instate satisfactory pain relief by programming one or more new programs according to the pre-defined workflow.

The conventional clinical standard consists in the clinical field specialist performing the programming session using a pre-defined workflow. Those sessions can be time-consuming and require the availability of the clinical field specialist and clinical space.

However, as a drawback of known solutions, therapy programming by a clinical field specialist of the device's company, either the first time after implant or during follow-ups, is the only existing solution to therapy programming. The drawbacks are the necessity of patient-clinical field specialist coordination for programming session, which depends on the schedule of each actor, and the programming time that can be extensive, reaching more than 1 h 30 of total session time. When programming is done in office, such long periods of time can be exhaustive for patients as well as schedule-consuming for clinical field specialists, who are then able to dedicate less time to other patients or tasks on a programming day.

An objective derived from these drawbacks may be
(1) to reduce both the programming session time and the clinical field specialist's working hours dedicated to just one patient and
(2) to allow remote self-programming and save the patient a systemic trip to the office for programming and
(3) to promote standardized therapy programming by following a very consistent workflow and collecting outcomes and thus further refine the workflow to increase chances of optimal pain relief as soon as possible.

Automatic programming uses a digital system that takes the patient's feedback as input and system information (such as position of the implanted leads, automatically detected position of the patient's body) and automatically programs or adjusts the therapy according to a predefined workflow. For instance, this system can take the form of an additional module in the patient's personal remote software, accessible by clicking on a dedicated button in the Patient Remote's app menu. The next steps may be as follows:
1. The patient triggers a programming session by clicking the button
2. The patient confirms with a personal PIN number or password (provided with the patient remote) that they want to start a programming session and they understand the function of it (some additional information is made available via a hyperlink in the dialog box).
3. The patient confirms their intention to start a programming session entering the type of session (e.g. initial programming, follow up due to unsatisfactory pain relief), made understandable by a brief question (e.g. “is it your first time turning on therapy?” Yes/No).
4. The programming session then starts just as a normal workflow followed by a clinical field specialist would, depending on the company's established programming method. But instead of interacting with a clinical field specialist, the latter is embodied by a bot that communicates with the patient via a chat or automatized call. As a programming session example:
   Optionally, the position of the implanted leads relative to each other (when there is more than one lead implanted) is automatically detected by the system and will be used as a factor to suggest therapy programs. Optionally, the position of the leads relative to the vertebrae is automatically detected or pulled from previous records and will be used as a factor to suggest therapy programs.
   Optionally, the patient's body position is either detected by a wearable or implanted sensor, or sensor included in the implanted device, or it is asked directly through the chat or automatized call. The patient's reply (by for example clicking the answer option “sitting” or “lying down” or “in a reclined chair”) is recorded for future use as an input to program definition or adjustment.
   Then, the bot asks the patient for their NRS. The chat is automatized in the most humane way, with dialogues mimicking what a real conversation between patient and clinical field specialist would sound like. The patient can click on certain text boxes, so that they don't even have to always type their answer (for example after a question, the chat displays a ‘Yes’ and ‘No’ in the form of clickable text boxes, and the patient just has to click one of them. If any typing error is made, they can just type ‘back’ to be asked the last question again and change their answer.
   In general, the chat or automatized call should be structured, both the form and the content, in the most intuitive way for the patient, yet with safety steps ensuring that the patient's input is what they really intended to provide, and minimizing the risks of transient overstimulation.
   In the present example, the patient should always have easy access to a ‘stop stimulation’ button and a ‘call clinical field specialist’ (or equivalent) buttons, ideally showing in the header of the chat window.
5. The system then starts paresthesia threshold search by turning on stimulation according to the pre-defined workflow, taking into account previous parameters if it is a follow-up, with patient-authorized stimulation onset and increases. It records paresthesia threshold by asking authorization to increase stimulation and subsequently asking if the patient felt something. Paresthesia threshold is found once the patient clearly felt stimulation, or however paresthesia threshold finding is defined in the pre-defined workflow (e.g. with Evoked Compound Action Potentials, eCAPs, detection)
6. Following paresthesia threshold finding, and according to the pre-defined workflow, the bot offers to program a first therapy based on all collected and saved inputs (e.g. NRS scores, pain map, paresthesia threshold, previous paresthesia threshold, pain scores and programs if it is a follow up). Once the patient accepts, the proposed program is uploaded to the patient's device.
7. Stimulation is turned on with patient's confirmed authorization (as before through clickable buttons) and amplitude is increased step-by-step with patient's authorization until paresthesia is found (when the patient feels stimulation), and then adjusted to a comfortable level (either sub-perception or with paresthesia depending on the program and patient's preference).
8. Optionally, if the patient's body position was recorded at the beginning of the programming session, the bot asks the patient to change position and indicate if they feel stimulation in a range of body positions different from during programming (e.g. if the patient was sitting during programming, the bot would ask the patient to lay down and stand up and asks if the patient felt something for each position), This allows the bot to refine the program parameters (e.g. amplitude) according to the pre-defined workflow.
9. Optionally, and according to the pre-defined workflow, the bot offers the patient to create one or several other programs so that the patient will have different therapies to easily choose from with their remote. The way the bot creates this or those programs is the same as the first program.
10. At the end of the programming session, or after each program is parametrized. the system uploads all new programs and settings as a result of the pre-defined workflow. The bot informs the patient that the patient should wait at least a pre-defined period of time (e.g. 24 hours) before evaluating the efficacy of the new programs and triggering a new programming session.

It is important to note that at any time, the patient can decide to call a clinical field specialist who can remotely take over the programming session just as it would be typically done in-office. The specialist would have access to all data collected and interactions made with the bot prior to the call, in order to get quickly updated with the programming session's status. This is made easy through an always visible button on the chat window, or with a key word if it is an automatized call (e.g. “talk to someone”).

The call to the clinical field specialist is handled via a patient support workflow of the company's choice. For example, the call can go directly to a clinical field specialist of choice. If they are not available, the call is re-directed to a e.g. patient support center where any clinical field specialist available at that moment can answer the call. If no one is available, an action item (notification) is flagged in the support center system so that as soon as a clinical field specialist is available, they can call the patient back.

An alternative patient support workflow restricts the call to a handful of clinical specialists assigned to the patient (or patient group in which the patient is classified) instead of a patient support center, and leaves a notification to all of them until one of them calls the patient back.

Embodiments of the inventive solution may provide for the following characteristics:
The non-human (automatized) nature of the programming process is one key to this invention.
The automated interactions between a non-human entity (bot, automatized voice call) and the patient following a pre-defined workflow to complete the programming session.
The ability of the automated programming session to collect patient's data required for therapy programming, such as the pain score (e.g. NRS), paresthesia threshold and body position through carefully pre-programmed interactions between the bot and the patient.
The ability to safely program therapy via patient-driven, discretized stimulation increases.
The intuitive, human-mimicking nature of the dialog created between the non-human entity and the patient, to maximize the positivity of the patient's experience.
The safeguard features of the process (confirmation buttons, ability to be taken back to the previous question, ability to stop stimulation or call a clinical field specialist at any time) to ensure the patient's safety.

Embodiments of the inventive solution may provide for the following advantages:
More consistency of programming sessions, that can lead to better understanding of programming outcomes and optimums, and thus allow refinement of the workflow to improve and/or accelerate pain relief.
Less bias in the patient's evaluation of pain and/or program efficacy due to human interactions.
Save the patient a trip to the office or lengthy programming session with a clinical field specialist.
Allow the patient to program or re-program their therapies at their convenience, without having to wait for the availability of a clinical field specialist or having to find a time slot that accommodates both actors.
Prevent the patient from having to wait hours or more when therapy is not working anymore (the patient feels pain at unacceptable levels again) for a programming session. With automated programming, they can start at any time, any day.
Save the clinical field specialist time to focus on other tasks and/or take on more patients.

Finally, it should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

### LIST OF REFERENCE NUMERALS

1 medical device system
3 implantable medical device
5 patient device
7 clinician device
9 body
10 spine
11 patient
13 neurostimulator
15 electrode
17 implantable lead
19 first interface
21 touchscreen
23 second interface
25 interface in the implantable medical device
27 third interface
29 clinician
31 touchscreen
33 fourth interface
35 clinician contact button
37 body position sensor
39 stop stimulation bottom
41 fifth interface

1. A medical device system (1), comprising:
(a) a programmable implantable medical device (3), configured for providing stimulations to a patient (11) in accordance with programmed stimulation parameters;
(b) a patient device (5), comprising a first interface (19) configured for communication and interaction with the patient (11) and a second interface (23) configured for submitting data for programming the implantable medical device (3); and
(c) a clinician device (7), comprising a third interface (27) configured for communication and interaction with a clinician (29),
wherein, for programming the implantable medical device (1), the patient device (5) is configured to automatically pose at least one question to the patient (11) via the first interface (19), and to automatically decide upon an action according to at least one feedback from the patient (11) entered via the first interface (19) and to submit the adjusted stimulation parameter to the implantable medical device (3), and wherein the patient device (5) and the clinician device (7) are further configured to provide an option to the patient (11) to selectively establish a communication between the patient (11) and the clinician (29).

2. The medical device system of claim 1, wherein the patient device (5) and the clinician device (7) are further configured to provide an option to the patient (11) to communicate with a clinician (29) by at least one of:
(i) establishing a data communication between the patient device (5) and the clinician device (7), or
(ii) providing a notification on the third interface (27) to the clinician (29) to call back the patient (11).

3. The medical device system of claim 1, wherein the clinician device (7) comprises a fourth interface (33), and wherein for establishing the communication between the patient (11) and the clinician (29);
(i) the patient device (5) is configured to setup a data communication to an external server, whereupon the external server is configured to setup a data communication to the clinician device (7) via the fourth interface (33), or
(ii) the clinician device (7) is configured to setup a data communication to an external server via the fourth interface (33), whereupon the external server is configured to setup a data communication to the patient device (5),
wherein the data communication between the patient device (5) and the clinician device (7) is carried out via the external server.

4. The medical device system of claim 1, wherein the patient device (5) and the clinician device (7) are further configured to provide an option to the clinician device (7) to establish access to data stored in the patient device (5) using the data communication between the patient device (5) and the clinician device (7).

5. The medical device system of claim 1, wherein the patient device (5) comprises a clinician contact button (35) configured for at least one of:
(i) establishing the communication between the patient (11) and the clinician (29) upon the clinician contact button (35) being actuated by the patient (11), or
(ii) providing a notification to the clinician (29) to call back the patient (11) on the third interface (27) upon the clinician contact button (35) being actuated by the patient (11).

6. The medical device system of claim 1, wherein the patient device (5) is configured to automatically pose the at least one question in a manner mimicking an inter-human communication.

7. The medical device system of claim 1, wherein the implantable medical device (3) comprises at least one implantable electrode (15), wherein the medical device system (1) is configured for automatically determining a position of the implantable electrode (15) relative to one of:
(i) the patient's body (9), or
(ii) a position of a neighboring electrode (15),
and wherein the patient device (5) is configured to at least one of:
(i) automatically posing pose the at least one question to the patient (11) in dependence of the determined position of the implantable electrode (15), or
(ii) automatically decide upon an action according to at least one feedback from the patient (11) in dependence of the determined position of the implantable electrode (15).

8. The medical device system of claim 1, wherein the medical device system (1) comprises at least one body position sensor (37), wherein the medical device system (1) is configured for automatically determining a position of the patient's body (9) based on signals provided by the body position sensor (37), and wherein the patient device (5) is configured to at least one of:
(i) automatically pose the at least one question to the patient (11) in dependence of the determined position of the patient's body (9), or
(ii) automatically adjust the at least one stimulation parameter according to at least one feedback from the patient (11) in dependence of the determined position of the patient's body (9).

9. The medical device system of claim 1, wherein the action comprises at least one of:
(i) an adjustment of at least one stimulation parameter,
(ii) a decision to not adjust any stimulation parameter,
(iii) initiating a call with the clinician device (7),
(iv) providing a notification to the clinician (29) to call back the patient (11) on the third interface (27), or
(v) pose posing another question to the patient (11) via the first interface (19).

10. The medical device system according to claim 9, wherein the patient device (5) is configured to submit the adjusted stimulation parameter to the implantable medical device (3) exclusively upon the patient (11) having authorized such submitting.

11. The medical device system of claim 1, wherein the patient device (5) comprises a stop stimulation button (39) configured for immediately instructing the implantable medical device (3) to stop providing stimulations to the patient (11) upon the stop stimulation button (39) being actuated by the patient (11).

12. Method A method for operating a medical device system (1), the medical device system (1) comprising:
(a) a programmable implantable medical device (3), configured for providing stimulations to a patient (11) in accordance with programmed stimulation parameters;
(b) a patient device (5), comprising a first interface (19) configured for communication and interaction with the patient (11) and a second interface (23) configured for submitting data for programming the implantable medical device (3); and
(c) a clinician device (7), comprising a third interface (27) configured for communication and interaction with a clinician (29),
wherein the method comprises:
(a) automatically posing at least one question to the patient (11) via the first interface (19) of the patient device (5);
(b) automatically decide upon an action according to at least one feedback from the patient (11) entered via the first interface (19); and
(c) submitting the adjusted stimulation parameter to the implantable medical device (3) for programming the implantable medical device (3),
wherein, during operation of the patient device (5), an option is provided to the patient (11) to selectively establish a communication between the patient (11) and the clinician (29).

13. The method of claim 12, wherein, upon specifically actuating the patient device (5) by the patient (11), an option is provided to the patient (11) to communicate with a clinician (29) by at least one of:
(i) establishing a data communication between the patient device (5) and the clinician device (7), or
(ii) providing a notification on the third interface (27) to the clinician to call back the patient (11).

14. The method of claim 11, wherein the clinician device (7) comprises a fourth interface (33), and wherein for establishing the communication between the patient (11) and the clinician (29);
(i) a data communication is set up between the patient device (5) and an external server, whereupon a data communication is set up between the external server to the clinician device (7) via the fourth interface (33), or
(ii) a data communication is set up between the clinician device (7) and an external server via the fourth interface (33), whereupon a data communication is set up between the external server to the patient device (5),
wherein the data communication between the patient device (5) and the clinician device (7) is carried out via the external server.

15. The method of ene of claim 12, wherein, during operation of the patient device (5), an option is provided to the clinician (29) to access to data stored in the patient device (5) using the data communication between the patient device (5) and the clinician device (7).
